(19) United States
(12) Patent Application Publication
(10) Pub. No.: US 2014/0294675 A1
(43) Pub. Date: Oct. 2, 2014

(54) Marker Detection Method And Apparatus To Monitor Drug Compliance

(71) Applicants: Richard J. Melker, Gainesville, FL (US); Donn Michael Dennis, Gainesville, FL (US); Laszlo Prokai, Gainesville, FL (US)

(72) Inventors: Richard J. Melker, Gainesville, FL (US); Donn Michael Dennis, Gainesville, FL (US); Laszlo Prokai, Gainesville, FL (US)

(73) Assignee: University of Florida Research Foundation, Inc., Gainesville, FL (US)

(21) Appl. No.: 14/230,043

(22) Filed: Mar. 31, 2014

Related U.S. Application Data
(63) Continuation of application No. 11/097,647, filed on Apr. 1, 2005.
Application No. 11/097,647 is a continuation-in-part of application No. 10/722,620, filed on Nov. 26, 2003, now Pat. No. 7,820,108.
Application No. 10/722,620 is a continuation of application No. 09/708,789, filed on Nov. 8, 2000, now abandoned.
(60) Provisional application No. 60/164,250, filed on Nov. 8, 1999.

(51) Int. Cl. G01N 33/497

(52) U.S. Cl. CPC G01N 33/497
USPC 422/84

(57) ABSTRACT

The present invention includes systems and methods for monitoring therapeutic drug concentration in blood by detecting markers, such as odors, upon exhalation by a patient after the drug is taken, wherein such markers result either directly from the drug itself or from an additive combined with the drug. In the case of olfactory markers, the invention preferably utilizes electronic sensor technology, such as the commercial devices referred to as “artificial” or “electronic” noses or tongues, to non-invasively monitor drug levels in blood. The invention further includes a reporting system capable of tracking drug concentrations in blood (remote or proximate locations) and providing the necessary alerts with regarding to ineffective or toxic drug dosages in a patient.

### CROSS-REFERENCE TO A RELATED APPLICATION

This application is a continuation of pending U.S. Ser. No. 11/097,647, filed on Apr. 1, 2005, which was a continuation-in-part application of pending U.S. Ser. No. 10/722,620, filed Nov. 26, 2003, which claims the benefit of U.S. Provisional Application No. 60/164,250, filed Nov. 8, 1999, and is a continuation application of U.S. Ser. No. 09/708,789, filed Nov. 8, 2000, now abandoned, all of which are hereby incorporated by reference herein in their entirety.

### FIELD OF INVENTION

The present invention relates to marker detection for monitoring patient compliance, and, more particularly, to a method and system for detecting in a bodily fluid sample markers associated with a therapeutic agent, wherein the markers are derived either from the therapeutic agent or from an additive combined with the therapeutic agent.

### BACKGROUND INFORMATION

Non-compliance of patients to drug regimens prescribed by their physicians results in excessive healthcare costs estimated to be around $100 billion per year through lost work days, increased cost of medical care, higher complication rates, as well as drug wastage. Non-compliance refers to the failure to take the prescribed dosage at the prescribed time which results in undermedication or overmedication. In a survey of 57 non-compliance studies, non-compliance ranged from 15% to as high as 95% in all study populations, regardless of medications, patient population characteristics, drug delivery method, or study methodology (Greenberg, R N, “Overview of patient compliance with medication dosing: A literature review,” Clinical Therapeutics, 6(5):592-599 (1984)).

The sub-optimal rates of compliance reported by various studies becomes of even greater concern as the American populace ages and becomes more dependent on drugs to fight illnesses accompanying old age. By 2025, over 17% of the US population will be over 65 (Bell, J A et al., “Clinical research in the elderly: Ethical and methodological considerations,” Drug Intelligence and Clinical Pharmacy, 21:1002-1007 (1987)) and senior citizens take, on average, over three times as many drugs compared to the under 65 population (Cosgrove, R, “Understanding drug abuse in the elderly,” Midwife, Health Visitor & Community Nursing, 24(6):222-223 (1988)). The forgetfulness that sometimes accompanies old age also makes it even more urgent to devise cost-effective methods of monitoring compliance on a large scale.

Further, non-compliance of patients with communicable diseases (e.g., tuberculosis and related opportunistic infections) costs the public health authorities millions of dollars annually and increases the likelihood of drug-resistance, with the potential for widespread dissemination of drug-resistant pathogens resulting in epidemics.

A cost-effective, but difficult to administer, program has been developed in seven locations around the nation to combat this serious threat to the American populace. It involves direct observation of all drug delivery by trained professionals (entitled “directly observed therapy” or “DOT”) but is impractical for large scale implementation. Many techniques are also invasive, e.g., blood, feces or urine sampling.

Accordingly, there is a need in the art for a method to improve drug compliance which provides simple monitoring of medication dosing which is non-invasive, intuitive and sanitary.

### SUMMARY OF THE INVENTION

The present invention solves the needs in the art by providing methods and systems for monitoring drug compliance by detecting markers, such as odors, presented in a patient after scheduled administration of a prescribed medication. Such markers result either directly from the therapeutic drug or from an additive combined with the drug. In the case of olfactory markers, the invention preferably utilizes electronic sensor technology, such as commercial devices referred to as “artificial noses” or “electronic noses,” to non-invasively monitor patient compliance. The invention further includes a reporting system capable of tracking compliance (remote or proximate) and providing the necessary alerts to monitoring personnel.

Therefore, it is an object of the present invention to detect marker substances in patient bodily fluids, wherein the marker is an indicator of patient compliance in taking a therapeutic agent as prescribed. Contemplated devices and methods for detecting such markers include, but are not limited to, devices commonly known as “artificial” or “electronic” noses or tongues, metal-insulator-metal ensemble (MIME) sensors, cross-reactive optical microsensor arrays, fluorescent polymer films, surface enhanced raman spectroscopy (SERS), semiconductor gas sensor technology, conductive polymer gas sensor technology, surface acoustic wave gas sensor technology, and immunoassays.

It is a further object of the present invention to provide a reporting system capable of tracking compliance based on marker detection and alerting patients, healthcare personnel, and/or in some instances health officials of patient non-compliance. In certain related embodiments, the reporting system provides necessary outputs, controls, and alerts to any one or all of the individuals listed above.

In one embodiment, a therapeutic agent is prescribed to a patient, wherein the therapeutic agent is to be taken by volitional patient action. Patient bodily fluid, preferably exhaled breath, is then analyzed by a sensor to detect a therapeutic drug marker that is indicative of therapeutic agent presence in the patient's biological system.

In certain embodiments, the therapeutic drug marker is the therapeutic agent itself. Accordingly, should a therapeutic agent be taken under volitional patient action, analysis of the patient's bodily fluid (such as exhaled breath) thereafter by a sensor of the invention will identify the presence of the therapeutic agent, which indicates patient compliance with a prescribed therapeutic regimen.

In related embodiments, the therapeutic drug marker is a metabolite of the therapeutic agent, wherein the metabolite is released for detection in patient bodily fluid after the therapeutic agent is metabolized or interacts with certain compounds or enzymes to produce or liberate the metabolite for detection.

In other embodiments, the therapeutic drug marker is an additive, or a metabolite of an additive, that is administered concurrently with a therapeutic agent to a patient. Preferably, the therapeutic agent and additive are metabolized by the patient. To assess whether the patient complies with the prescribed therapeutic drug regimen, a sample of the patient's bodily fluid (e.g., exhaled breath) is analyzed to detect the therapeutic drug marker, which (in these embodiments) is either the additive or a metabolite of the additive, or a combination of the two.

In one preferred embodiment, the therapeutic drug marker is at least one GRAS (Generally Recognized As Safe) compound and/or a metabolite of the GRAS compound, that is detectable in exhaled breath using a sensor of the invention. A GRAS compound, as used herein, refers to additives approved by the U.S. Food and Drug Administration Center for Food Safety and Applied Nutrition.

More preferably, a specific phase of the respiratory cycle, namely the end-tidal portion of exhaled breath, is sampled to detect the presence and/or concentration of a therapeutic drug marker as an indication of patient compliance with a prescribed therapeutic drug regimen.

A sensor of the subject invention would be used either in a clinical setting or patient-based location (such as the patient's home) after prescribed delivery of a therapeutic drug to monitor drug concentration in blood by measuring therapeutic drug marker concentration in patient exhaled breath. In cases where exhaled breath is analyzed, the systems and methods of the present invention enable accurate evaluation of pharmacodynamics and pharmacokinetics in individual patients and/or for drug studies.

Therefore, it is an object of the present invention to non-invasively monitor patient compliance with a prescribed regimen by monitoring therapeutic drug marker concentrations in exhaled breath using sensors that can detect markers in bodily fluids, especially exhaled breath. A resulting advantage of the subject invention is the ability to monitor such patient compliance in a more cost effective and frequent manner than current methods, which can involve the expensive and invasive procedure of drawing blood samples and transferring the blood samples to a laboratory facility for analysis.

The invention will now be described, by way of example and not by way of limitation, with reference to the accompanying sheets of drawings and other objects, features and advantages of the invention will be apparent from the following detailed disclosure and from the appended claims.

### BRIEF DESCRIPTION OF THE DRAWINGS

FIG. 1 shows a capnogram of a single respiratory cycle and a capnogram of several breaths from a patient with obstructive lung disease.

FIG. 2 shows a gas sensor chip which may be utilized as the sensor for the present invention.

FIG. 3 shows an overview of the preferred steps of the method of the present invention.

FIG. 4 shows the patient taking medication with a marker which is released for detection.

FIG. 5 shows the preferred marker detection system utilizing sensor technology which can communicate with a computer for proximate or remote monitoring.

FIG. 6 is a schematic diagram of CYP 3A4 metabolizing verapamil analogue to liberate a marker easily detected in exhaled breath.

FIG. 7 is a schematic diagram of CYP 2D6 metabolizing dextromethorphan analogue to liberate a marker easily detected in exhaled breath.

### DETAILED DESCRIPTION OF THE INVENTION

The present invention provides methods and apparatuses for monitoring patient compliance with a prescribed therapeutic drug regimen. After a therapeutic drug is prescribed to a patient, wherein the drug is to be taken by volitional patient action, patient bodily fluid is analyzed for the presence of a therapeutic drug marker, wherein the marker indicates therapeutic agent presence in the patient's biological system.

The therapeutic drug markers of the invention are derived either directly from medication comprising the therapeutic agent or from an additive combined with the medication. Such markers preferably include volatile or olfactory markers (odors) as well as other substances and compounds that may be detectable by various methods, as described in more detail herein.

In accordance with the subject invention, the marker is detected by devices including but not limited to electronic noses, spectrophotometers to detect the marker's IR, UV, or visible absorbance or fluorescence, or mass spectrometers to detect the marker's characteristic mass display.

In certain embodiments, the therapeutic drug is administered to a patient by a health care provider. Preferably, the therapeutic drug is administered by the patient to him (or her) self (also referred to herein as volitional patient action) in accordance with a therapeutic drug prescription regimen. A sample of the patient's bodily fluid (such as exhaled breath) is then analyzed to detect the presence of the therapeutic drug marker, which is an indication of patient compliance or non-compliance in taking the therapeutic drug.

### DEFINITIONS

As used herein, the term “therapeutic agent” or “therapeutic drug” refers to a substance used in the diagnosis, treatment, or prevention of a disease or condition. In one embodiment of the invention, the concentration of a therapeutic agent or drug in a patient's blood stream is monitored to ensure the therapeutic drug level is within a clinically effective range.

Throughout this disclosure, a “marker” or “therapeutic drug marker” is defined as a substance that is detected by means of its physical or chemical properties using a sensor of the subject invention. In certain embodiments, the therapeutic drug marker is non-toxic to the patient. The following are markers that can be detected in exhaled breath in accordance with the subject invention: a therapeutic drug, a metabolite of a therapeutic drug, an additive that is concurrently administered with a therapeutic drug, or a metabolite of an additive that is administered concurrently with a therapeutic drug. Preferred therapeutic drug markers include volatile and/or olfactory markers (odors) as well as other substances and compounds, which may be detectable by sensors of the subject invention.

A “patient,” as used herein, describes an organism, including mammals, from which exhaled breath samples are collected in accordance with the present invention. Mammalian species that benefit from the disclosed systems and methods for therapeutic drug monitoring include, and are not limited to, apes, chimpanzees, orangutans, humans, monkeys; and domesticated animals (e.g., pets) such as dogs, cats, mice, rats, guinea pigs, and hamsters.

The term “bodily fluid,” as used herein, refers to a mixture of molecules obtained from a patient. Bodily fluids include, but are not limited to, exhaled breath, whole blood, blood plasma, urine, semen, saliva, lymph fluid, meningeal fluid, amniotic fluid, glandular fluid (for example, breast milk), sputum, feces, sweat, mucous, vaginal fluid, ocular humors, and cerebrospinal fluid. Bodily fluid also includes experimentally separated fractions of all of the preceding solutions or mixtures containing homogenized solid material, such as feces, tissues, and biopsy samples.

The term “pharmacodynamics,” as used herein, refers to the interaction (biochemical and physiological) of a therapeutic drug with constituents of a patient body as well as the mechanisms of drug action on the patient body (i.e., drug effect on body).

As used herein, the term “pharmacokinetics” refers to the mathematical characterization of interactions between normal physiological processes and a therapeutic drug over time (i.e., body effect on drug). Certain physiological processes (absorption, distribution, metabolism, and elimination) will affect the ability of a drug to provide a desired therapeutic effect in a patient. Knowledge of a drug's pharmacokinetics aids in interpreting drug blood stream concentration and is useful in determining pharmacologically effective drug dosages.

“Concurrent” administration, as used herein, refers to the administration of a therapeutic drug marker with a therapeutic agent in accordance with the systems and methods of the invention for monitoring patient compliance with a prescribed regimen. By way of example, a therapeutic drug marker can be provided as an additive in admixture with a therapeutic drug, such as in a pharmaceutical composition/medication; or the marker and therapeutic drug can be administered to a patient as separate compounds, such as, for example, separate pharmaceutical compositions administered consecutively, simultaneously, or at different times. Preferably, if the marker and the therapeutic drug are administered separately, they are administered within sufficient time from each other so that the concentration of the marker in exhaled breath is an accurate indicator of the concentration of the therapeutic drug in the patient's blood stream.

The term “aptamer,” as used herein, refers to a non-naturally occurring oligonucleotide chain that has a specific action on a therapeutic drug marker. Aptamers include nucleic acids that are identified from a candidate mixture of nucleic acids. In a preferred embodiment, aptamers include nucleic acid sequences that are substantially homologous to the nucleic acid ligands isolated by the SELEX method. Substantially homologous is meant a degree of primary sequence homology in excess of 70%, most preferably in excess of 80%.

The “SELEX™” methodology, as used herein, involves the combination of selected nucleic acid ligands, which interact with a target marker in a desired action, for example binding to an olfactory marker, with amplification of those selected nucleic acids. Optional iterative cycling of the selection/amplification steps allows selection of one or a small number of nucleic acids, which interact most strongly with the target marker from a pool, which contains a very large number of nucleic acids. Cycling of the selection/amplification procedure is continued until a selected goal is achieved. The SELEX methodology is described in the following U.S. patents and patent applications: U.S. patent application Ser. No. 07/536,428 and U.S. Pat. Nos. 5,475,096 and 5,270,163.

As used herein, the term “pharmaceutically acceptable carrier” means a carrier that is useful in preparing a pharmaceutical composition that is generally compatible with the other ingredients of the composition, not deleterious to the patient, and neither biologically nor otherwise undesirable, and includes a carrier that is acceptable for veterinary use as well as human pharmaceutical use. “A pharmaceutically acceptable carrier” as used in the specification and claims includes both one and more than one such carrier.

### Breath Sampling

Generally, the exhalation gas stream comprises sequences or stages. At the beginning of exhalation there is an initial stage, the gas representative thereof coming from an anatomically inactive (deadspace) part of the respiratory system, in other words, from the mouth and upper respiratory tracts. This is followed by a plateau stage. Early in the plateau stage, the gas is a mixture of deadspace and metabolically active gases. The last portion of the exhaled breath comprises nothing but deep lung gas, so-called alveolar gas. This gas, which comes from the alveoli, is termed end-tidal gas.

In a preferred embodiment, the exhaled breath sample is collected at end-tidal breathing. Technology similar to that used for end-tidal carbon dioxide monitoring can be used to determine when the sample is collected. Known methods for airway pressure and airway flow measurements afford other means of collecting samples at the appropriate phase of the respiratory cycle. Single or multiple samples collected by the known side stream method are preferable, but if sensor acquisition time is reduced, in-line sampling may be used. In the former, samples are collected through an adapter at the proximal end of an endotracheal (ET) tube and drawn through thin bore tubing to a sensor of the subject invention.

Depending on the sample size and sensor response time, exhaled gas may be collected on successive cycles. With in-line sampling, a sensor of the subject invention is placed at the proximal end of the ET tube directly in the gas stream. Alternatively to sample end-tidal gas, samples can be taken throughout the exhalation phase of respiration and an average value determined and correlated with blood concentration.

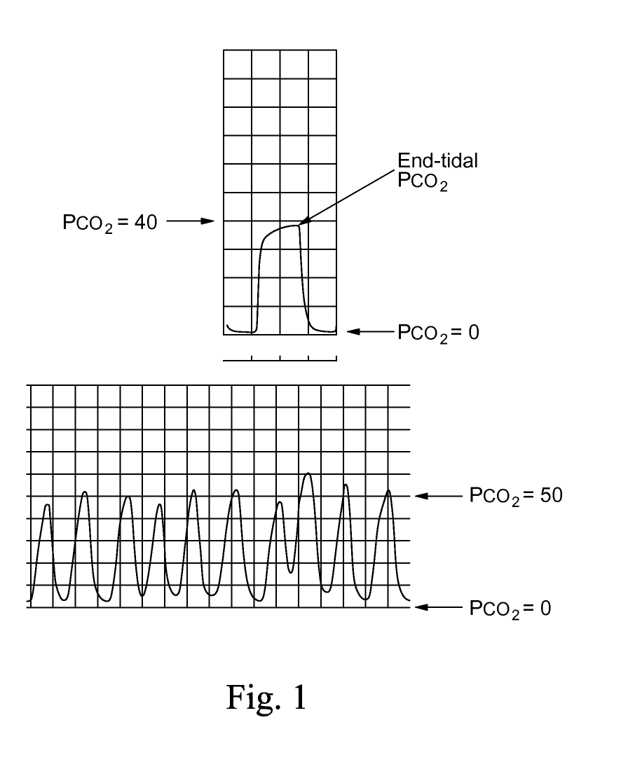

Referring now to FIG. 1, the upper frame demonstrates a capnogram of a single respiratory cycle. For accurate blood level correlation, samples are taken at the point labeled “end-tidal PCO₂” which reflects the CO₂ concentration in the lung. The lower frame shows a capnogram of several breaths from a patient with obstructive lung disease. Again the end-tidal sample correlated best with blood concentration.

In one embodiment, a VaporLab™ brand instrument is used to collect and analyze exhaled breath samples. The VaporLab™ instrument is a hand-held, battery powered SAW-based chemical vapor identification instrument suitable for detecting components in exhaled breath samples in accordance with the present invention. This instrument is sensitive to volatile and semi-volatile compounds using a high-stability SAW sensor array that provides orthogonal vapor responses for greater accuracy and discrimination. In a related embodiment, this instrument communicates with computers to provide enhanced pattern analysis and report generation. In a preferred embodiment, this instrument includes neural networks for “training” purposes, i.e., to remember chemical vapor signature patterns for fast, “on-the-fly” analysis.

In another embodiment, samples are collected at the distal end of an ET through a tube with a separate sampling port. This may improve end-tidal sampling.

In certain instances, the concentration of a therapeutic drug in a patient body is regulated by the amount of the drug administered over a given time period and the rate at which the agent is eliminated from the body (metabolism). Examples of how therapeutic drugs and/or therapeutic drug markers are eliminated or metabolized by the body include, but are not limited to, metabolism by stomach acid (such as therapeutic drug marker interaction with stomach acid such that the therapeutic drug marker is detectable in exhaled breath); or absorption into the body (such as absorption into the gastro-intestinal tract) and metabolized by cells in the body to release a therapeutic drug marker that is detectable in exhaled breath.

The present invention provides the steps of administering a therapeutic drug to a patient and analyzing patient exhaled breath for concentration of therapeutic drug markers such as unbound substances, active metabolites, or inactive metabolites associated with either the therapeutic drug or an additive, after a suitable time period. In certain embodiments of the subject invention, the marker concentration indicates a characteristic of metabolism of the drug in the patient.

Methods of the subject invention may further include the use of a flow sensor to detect starting and completion of exhalation. The method further includes providing results from the analysis and communicating to the user or patient the blood concentration of the therapeutic drug. Moreover, a CPU may be provided as a data processing/control unit for automatically detecting the signal from the flow sensor to control sampling of exhaled breath. The CPU may further provide to the user/patient the appropriate dosage of the therapeutic drug to be delivered based on analysis of trends in therapeutic drug blood concentration.

In another embodiment, the exhalation air is measured for marker concentration either continuously or periodically. From the exhalation air is extracted at least one measured marker concentration value. Numerous types of breath sampling apparatuses can be used to carry out the method of the present invention.

In one embodiment, the breath sampling apparatus includes a conventional flow channel through which exhalation air flows. The flow channel is provided with a sensor of the subject invention for measuring marker concentration. Furthermore, necessary output elements may be included with the breath sampling apparatus for delivering at least a measured concentration result to the user, if necessary.

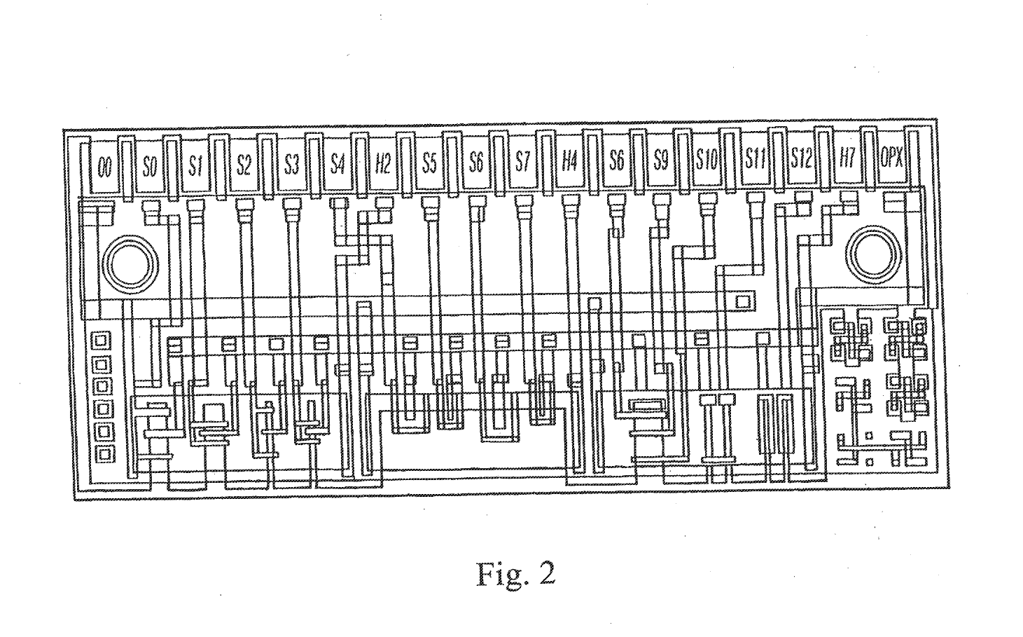

An alarm mechanism may also be provided. An instrument of similar type is shown in FIGS. 1 and 2 of U.S. Pat. No. 5,971,937 incorporated herein by reference.

### Sensor Technology

The invention preferably utilizes gas sensor technology, such as commercial devices known as “artificial” or “electronic” tongues or noses, to non-invasively monitor marker concentration in exhaled breath (FIG. 2). Electronic noses have been used mostly in the food, wine, and perfume industry where their sensitivity makes it possible to distinguish between odorous compounds. For example, electronic noses have been useful in distinguishing between grapefruit oil and orange oil in the perfume industry and identify spoilage in perishable foods before the odor is evident to the human nose.

In the past, there was little medical-based research and application of these artificial/electronic tongues and noses. However, recent use has demonstrated the power of this non-invasive technique. For example, electronic noses have been used to determine the presence of bacterial infection in the lungs by analyzing the exhaled gases of patients for odors specific to particular bacteria (Hanson C W, Steinberger H A, “The use of a novel electronic nose to diagnose the presence of intrapulmonary infection,” Anesthesiology, 87(3A):Abstract A269, (1997)). Also, a genitourinary clinic has utilized an electronic nose to screen for, and detect bacterial vaginosis, with a 94% success rate after training (Chandiok S, et al., “Screening for bacterial vaginosis: a novel application of artificial nose technology,” Journal of Clinical Pathology, 50(9):790-1 (1997)). Specific bacterial species can also be identified with the electronic nose based on special odors produced by the organisms (Parry A D et al., “Leg ulcer odor detection identifies beta-haemolytic streptococcal infection,” Journal of Wound Care, 4:404-406 (1995)).

A number of patents which describe gas sensor technology that can be used in the subject invention include, but are not limited to, the following: U.S. Pat. Nos. 5,945,069; 5,918,257; 4,938,928; 4,992,244; 5,034,192; 5,071,770; 5,145,645; 5,252,292; 5,605,612; 5,756,879; 5,783,154; and 5,830,412. Other sensors suitable for the present invention include, but are not limited to, metal-insulator-metal ensemble (MIME) sensors, cross-reactive optical microsensor arrays, fluorescent polymer films, surface enhanced raman spectroscopy (SERS), diode lasers, selected ion flow tubes, metal oxide sensors (MOS), bulk acoustic wave sensors, colorimetric tubes, infrared spectroscopy.

Recent developments in the field of detection that can also be used as sensor for the subject invention include, but are not limited to, gas chromatography, semiconductive gas sensors, mass spectrometers (including proton transfer reaction mass spectrometry), and infrared (IR) or ultraviolet (UV) or visible or fluorescence spectrophotometers (i.e., non-dispersive infrared spectrometer). For example, with semiconductive gas sensors, markers cause a change in the electrical properties of semiconductor(s) by making their electrical resistance vary, and the measurement of these variations allows one to determine the concentration of marker(s). In another example, gas chromatography, which consists of a method of selective detection by separating the molecules of gas compositions, may be used as a means for analyzing markers in exhaled breath samples.

In accordance with the subject invention, sensors for detecting/quantifying markers utilize a relatively brief detection time of around a few seconds. Other recent gas sensor technologies contemplated by the present invention include apparatuses having conductive-polymer gas-sensors (“polymeric”), aptamer biosensors, amplifying fluorescent polymer (AFP) sensors, apparatuses having surface-acoustic-wave (SAW) gas-sensors, photo-ionization detectors, and ion mobility spectrometry.

The conductive-polymer gas-sensors (also referred to as “chemoresistors”) have a film made of a conductive polymer sensitive to the molecules of odorous substances. On contact with target marker molecules, the electric resistance of the sensors changes and the measurement of the variation of this resistance enables identification of the target marker and its concentration. An advantage of this type of sensor is that it functions at temperatures close to room temperature. Different sensitivities for detecting different markers can be obtained by modifying or choosing an alternate conductive polymer.

Polymeric gas sensors can be built into an array of sensors, where each sensor is designed to respond differently to different markers and augment the selectivity of the therapeutic drug markers. For example, a sensor of the subject invention can comprise of an array of polymers, (i.e., 32 different polymers) each exposed to a marker. Each of the individual polymers swells differently to the presence of a marker, creating a change in the resistance of that membrane and generating an analog voltage in response to that specific marker (“signature”). The normalized change in resistance can then be transmitted to a processor to identify the type, quantity, and quality of the marker based on the pattern change in the sensor array. The unique response results in a distinct electrical fingerprint that is used to characterize the marker. The pattern of resistance changes of the array is diagnostic of the marker in the sample, while the amplitude of the pattern indicates the concentration of the marker in the sample.

Another sensor of the invention can be provided in the form of an aptamer. In one embodiment, the SELEX™ (Systematic Evolution of Ligands by Exponential enrichment) methodology is used to produce aptamers that recognize therapeutic drug markers with high affinity and specificity. Aptamers produced by the SELEX methodology have a unique sequence and the property of binding specifically to a desired marker. The SELEX methodology is based on the insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. According to the subject invention, therapeutic drug markers of any size or composition can thus serve as targets for aptamers. See also Jayasena, S., “Aptamers: An Emerging Class of Molecules That Rival Antibodies for Diagnostics,” Clinical Chemistry, 45:9, 1628-1650 (1999).

Aptamer biosensors can be utilized in the present invention for detecting the presence of markers in exhaled breath samples. In one embodiment, aptamer sensors are composed of resonant oscillating quartz sensors that can detect minute changes in resonance frequencies due to modulations of mass of the oscillating system, which results from a binding or dissociation event (i.e., binding with a target therapeutic drug marker).

Similarly, amplifying fluorescent polymer (AFP) sensors may be utilized in the present invention for detecting the presence of therapeutic drug markers in exhaled breath samples. AFP sensors are extremely sensitive and highly selective chemosensors that use amplifying fluorescent polymers. When vapors bind to thin films of the polymers, the fluorescence of the film decreases. A single molecule binding event quenches the fluorescence of many polymer repeat units, resulting in an amplification of the quenching. The binding of markers to the film is reversible, therefore the films can be reused.

Surface-acoustic-wave (SAW) sensors oscillate at high frequencies and generally have a substrate, which is covered by a chemoselective material. In SAW sensors, the substrate is used to propagate a surface acoustic wave between sets of interdigitated electrodes (i.e., to form a transducer). The chemoselective material is coated on the transducer. When a marker interacts with the chemoselective material coated on the substrate, the interaction results in a change in the SAW properties, such as the amplitude of velocity of the propagated wave. The detectable change in the characteristic wave is generally proportional to the mass load of the marker(s) (i.e., concentration of the marker in exhaled breath, which corresponds to the concentration of the therapeutic drug in the blood stream).

Certain embodiments of the invention use known SAW devices, such as those described in U.S. Pat. Nos. 4,312,228 and 4,895,017, and Groves W. A. et al., “Analyzing organic vapors in exhaled breath using surface acoustic wave sensor array with preconcentration: Selection and characterization of the preconcentrator adsorbent,” Analytica Chimica Acta, 371:131-143 (1988). Other types of chemical sensors known in the art that use chemoselective coating applicable to the operation of the present invention include bulk acoustic wave (BAW) devices, plate acoustic wave devices, interdigitated microelectrode (IME) devices, optical waveguide (OW) devices, electrochemical sensors, and electrically conducting sensors.

In one embodiment, the sensor of the invention is based on surface acoustic wave (SAW) sensors. The SAW sensors preferably include a substrate with piezoelectric characteristics covered by a polymer coating, which is able to selectively absorb target markers. SAW sensors oscillate at high frequencies and respond to perturbations proportional to the mass load of certain molecules. This occurs in the vapor phase on the sensor surface.

In a related embodiment, the SAW sensor is connected to a computer, wherein any detectable change in frequency can be detected and measured by the computer. In a preferred embodiment, an array of SAW sensors (4-6) is used, each coated with a different chemoselective polymer that selectively binds and/or absorbs vapors of specific classes of molecules. The resulting array, or “signature” identifies specific compounds.

The operating performance of most chemical sensors that use a chemoselective film coating is greatly affected by the thickness, uniformity and composition of the coating. For these sensors, increasing the coating thickness, has a detrimental effect on the sensitivity. Only the portion of the coating immediately adjacent to the transducer/substrate is sensed by the transducer.

For example, if the polymer coating is too thick, the sensitivity of a SAW device to record changes in frequency will be reduced. These outer layers of coating material compete for the marker with the layers of coating being sensed and thus reduce the sensitivity of the sensor. Uniformity of the coating is also a critical factor in the performance of a sensor that uses a chemoselective coating since changes in average surface area greatly effect the local vibrational signature of the SAW device. Therefore, films should be deposited that are flat to within 1 nm with a thickness of 15-25 nm. In this regard, it is important not only that the coating be uniform and reproducible from one device to another, so that a set of devices will all operate with the same sensitivity, but also that the coating on a single device be uniform across the active area of the substrate.

If a coating is non-uniform, the response time to marker exposure and the recovery time after marker exposure are increased and the operating performance of the sensor is impaired. The thin areas of the coating respond more rapidly to a target marker than the thick areas. As a result, the sensor response signal takes longer to reach an equilibrium value, and the results are less accurate than they would be with a uniform coating.

Most current technologies for creating large area films of polymers and biomaterials involve the spinning, spraying, or dipping of a substrate into a solution of the macromolecule and a volatile solvent. These methods coat the entire substrate without selectivity and sometimes lead to solvent contamination and morphological inhomogeneities in the film due to non-uniform solvent evaporation. There are also techniques such as microcontact printing and hydrogel stamping that enable small areas of biomolecular and polymer monolayers to be patterned, but separate techniques like photolithography or chemical vapor deposition are needed to transform these films into microdevices.

Other techniques such as thermal evaporation and pulsed laser ablation are limited to polymers that are stable and not denatured by vigorous thermal processes. More precise and accurate control over the thickness and uniformity of a film coating may be achieved by using pulsed laser deposition (PLD), a physical vapor deposition technique that has been developed recently for forming ceramic coatings on substrates. By this method, a target comprising the stoichiometric chemical composition of the material to be used for the coating is ablated by means of a pulsed laser, forming a plume of ablated material that becomes deposited on the substrate.

Polymer thin films, using a new laser based technique developed by researchers at the Naval Research Laboratory called Matrix Assisted Pulsed Laser Evaporation (MAPLE), have recently been shown to increase sensitivity and specificity of chemoselective Surface Acoustic Wave vapor sensors. A variation of this technique, Pulsed Laser Assisted Surface Functionalization (PLASF) is preferably used to design compound specific biosensor coatings with increased sensitivity for the present invention. PLASF produces similar thin films for sensor applications with bound receptors for biosensor applications. By providing improved SAW biosensor response by eliminating film imperfections induced by solvent evaporation and detecting molecular attachments to specific target markers, high sensitivity and specificity is possible.

Certain extremely sensitive, commercial off-the-shelf (COTS) electronic noses, such as those provided by Cyrano Sciences, Inc. (“CSI”) (i.e., CSI's Portable Electronic Nose and CSI's Nose-Chip integrated circuit for odor-sensing, see U.S. Pat. No. 5,945,069—FIG. 1), may be used in the system and method of the present invention to monitor the exhaled breath from a patient. These devices offer minimal cycle time, can detect multiple markers, can work in almost any environment without special sample preparation or isolation conditions, and do not require advanced sensor design or cleansing between tests.

Photo-Ionization Detectors (PIDs) rely on the fact that all elements and chemicals can be ionized. The energy required to displace an electron and ‘ionize’ a gas is called its Ionization Potential (IP), measured in electron volts (eV). A PID uses an ultraviolet (UV) light source to ionize the gas. The energy of the UV light source must be at least as great as the IP of the sample gas. For example, benzene has an IP of 9.24 eV, while carbon monoxide has an IP of 14.01 eV. For the PID to detect the benzene, the UV lamp must have at least 9.24 eV of energy. If the lamp has an energy of 15 eV, both the benzene and the carbon monoxide would be ionized. Once ionized, the detector measures the charge and converts the signal information into a displayed concentration. Unfortunately, the display does not differentiate between the two gases, and simply reads the total concentration of both summed together.

Three UV lamp energies are commonly available: 9.8, 10.6 and 11.7 eV. Some selectivity can be achieved by selecting the lowest energy lamp while still having enough energy to ionize the gases of interest. The largest group of compounds measured by a PID is the organic group of compounds (compounds containing carbon), and they can typically be measured to parts per million (ppm) concentrations. PIDs do not measure any gases with an IP greater than 11.7 eV, such as nitrogen, oxygen, carbon dioxide and water vapor. The CRC Press Handbook of Chemistry and Physics includes a table listing the IPs for various gases. PIDs are sensitive (low ppm), low cost, fast responding, portable detectors. They also consume little power.

Ion Mobility Spectrometry (IMS) separates ionized molecular samples on the basis of their transition times when subjected to an electric field in a tube. As the sample is drawn into the instrument, it is ionized by a weak radioactive source. The ionized molecules drift through the cell under the influence of an electric field. An electronic shutter grid allows periodic introduction of the ions into the drift tube where they separate based on charge, mass, and shape. Smaller ions move faster than larger ions through the drift tube and arrive at the detector sooner. The amplified current from the detector is measured as a function of time and a spectrum is generated. A microprocessor evaluates the spectrum for the target marker compound, and determines the concentration based on the peak height.

IMS is an extremely fast method and allows near real time analysis. It is also very sensitive, and should be able to measure all markers of interest. IMS is moderate in cost (several thousand dollars) and larger in size and power consumption.

In one embodiment, the device of the present invention may be designed so that patients can exhale via the mouth or nose directly onto a sensor of the invention. In another embodiment, a patient's breath sample can be captured in a container (vessel) for later analysis using a sensor of the subject invention (i.e., mass spectrometer).

Results from sensor technology analysis of patient bodily fluid samples are optionally provided to the user (or patient) via a reporting means. In one embodiment, the sensor technology includes the reporting means. Contemplated reporting means include a computer processor linked to the sensor technology in which electronic or printed results can be provided. Alternatively, the reporting means can include a digital display panel, transportable read/write magnetic media such as computer disks and tapes which can be transported to and read on another machine, and printers such as thermal, laser or ink-jet printers for the production of a printed report.

The reporting means can provide the results to the user (or patient) via facsimile, electronic mail, mail or courier service, or any other means of safely and securely sending the report to the patient. Interactive reporting means are also contemplated by the present invention, such as an interactive voice response system, interactive computer-based reporting system, interactive telephone touch-tone system, or other similar system. The report provided to the user (or patient) may take many forms, including a summary of analyses performed over a particular period of time or detailed information regarding a particular bodily fluid sample analysis. Results may also be used to populate a financial database for billing the patient, or for populating a laboratory database or a statistical database.

A data monitor/analyzer can compare a pattern of response to previously measured and characterized responses from known markers. The matching of those patterns can be performed using a number of techniques, including neural networks. By comparing the analog output from each of the 32 polymers to a “blank” or control, for example, a neural network can establish a pattern that is unique to that marker and subsequently learns to recognize that marker. The particular resistor geometries are selected to optimize the desired response to the target marker being sensed. The sensor of the subject invention is preferably a self-calibrating polymer system suitable for detecting and quantifying markers in gas phase biological solutions to assess and/or monitor a variety of therapeutic drug markers simultaneously.

According to the subject invention, the sensor can include a programmable apparatus (such as a computer) that communicates therewith, which can also notify the medical staff and/or the patient as to any irregularities in dosing, dangerous drug interactions, and the like. This system will enable determination as to whether a patient has been administered a pharmacologically effective amount of a therapeutic drug. The device could also alert the patient (or user) as to time intervals and/or dosage of therapeutic drug to be administered as prescribed. This is especially useful for elderly patients who are often forgetful.

The sensor of the present invention might include integrated circuits (chips) manufactured in a modified vacuum chamber for Pulsed Laser Deposition of polymer coatings. It will operate the simultaneous thin-film deposition wave detection and obtain optimum conditions for high sensitivity of SAW sensors. The morphology and microstructure of biosensor coatings will be characterized as a function of process parameters.

The sensor used in the subject invention may be modified so that the bodily fluid to be analyzed is a sample of exhaled breath. In these embodiments, patients can exhale directly onto the sensor, without needing a breath sampling apparatus. For example, a mouthpiece or nosepiece will be provided for interfacing a patient with the device to readily transmit the exhaled breath to the sensor (See, i.e., U.S. Pat. No. 5,042,501). In a related embodiment, wherein the sensor is connected to a neural network, the output from the neural network is similar when the same patient exhales directly into the device and when the exhaled gases are allowed to dry before they are sampled by the sensor.

The humidity in the exhaled gases represents a problem for certain electronic nose devices (albeit not SAW sensors) that only work with “dry” gases. When using such humidity sensitive devices, the present invention may adapt such electronic nose technology so that a patient can exhale directly into the device with a means to dehumidify the samples. This is accomplished by including a commercial dehumidifier or a heat moisture exchanger (HME), a device designed to prevent desiccation of the airway during ventilation with dry gases.

Alternatively, the patient may exhale through their nose, which is an anatomical, physiological dehumidifier to prevent dehydration during normal respiration. Alternatively, the sensor device can be fitted with a preconcentrator, which has some of the properties of a GC column. The gas sample is routed through the preconcentrator before being passed over the sensor array. By heating and volatilizing the gases, humidity is removed and the marker being measured (analyte) can be separated from potential interferents.

Preferably, in operation, the sensor will be used to identify a baseline spectrum for the patient prior to drug administration, if necessary. This will prove beneficial for the detection of more than one therapeutic drug if the patient receives more than one drug at a time and possible interference from different foods and odors in the stomach, mouth, esophagus and lungs.

In certain embodiments, the therapeutic drug marker is detected via dermal analysis. For example, markers can be non-invasively detected using mid-infrared (“MIR”). With MIR, penetration of skin by IR ranges in only a few micrometers. Thus, because of this small penetration depth, the sensor device can detect the presence of the marker from the mixture of oils and sweat that is pumped to the skin surface.

Optochemical sensors (e.g., colorimetric strips) are based on changes in some optical parameter due to enzyme reactions or antibody-antigen bonding at a transducer interface. Such sensors may include enzyme optrodes and optical immunosensors and may also include different monitoring processes such as densitometric, refractometric or calorimetric devices. Thus, optochemical sensors are used in certain embodiments of the invention to detect markers via transdermal analysis.

In other embodiments, the therapeutic drug marker is detected from a sample of urine or blood. For example, a closed container is used to hold the sample of urine or blood, wherein a “headspace” is created in the container for the presence of any volatile therapeutic markers in the bodily fluid sample. A sensor of the invention is then applied to the headspace of the container to identify whether therapeutic drug markers are present, and if so, that the patient has complied with a prescribed regimen.

The headspace can be assessed using any of the sensor devices described herein, including conventional quantitative/analytic devices including, but not limited to, liquid chromatography-mass spectroscopy (LS-MS) or gas chromatography-mass spectroscopy (GC-MS). In a preferred embodiment, blood or urine samples are placed in vials and incubated at 98° F. An electronic nose is then applied to the headspace to measure the amount of marker present in the headspace.

### Therapeutic Drug Markers

According to the subject invention, upon administration of a therapeutic drug, detection of a therapeutic drug marker can occur under three distinct circumstances. In one, the therapeutic drug marker can “coat” or persist in the mouth, esophagus and/or stomach upon administration (i.e., with oral ingestion) and be detected upon exhalation (similar to the taste or flavor that remains in the mouth after eating a breath mint or drinking a liquid medication).

In a second instance, the therapeutic drug marker may be released for detection after therapeutic drug and/or additive reaction in the mouth or stomach with acid or enzymes that produce or liberate the marker for detection in a bodily fluid sample (such as exhaled breath).

Thirdly, the therapeutic drug marker can be made detectable in bodily fluids after the therapeutic agent and/or additive is metabolized. For example, the therapeutic agent can be absorbed in the gastrointestinal tract (and, in certain instances, metabolized in the patient's body) so that a detectable metabolite of the drug (or metabolite of the additive) is excreted in the lungs for notification that the therapeutic drug has been taken by the patient.

In a preferred embodiment, patient exhaled breath is analyzed for a therapeutic drug marker. More preferably, the therapeutic drug marker is both an additive and a metabolite of the additive, where the additive is concurrently administered to the patient with the therapeutic agent. In a related embodiment, a therapeutic drug marker such as naltrexone is concurrently administered with a therapeutic agent to a patient. A specific metabolite of naltrexone is volatile and detectable in exhaled breath.

In operation, certain embodiments of the invention contemplate detecting a marker that is derived or released after therapeutic drug or additive interaction with saliva, stomach acid, enzymes, or any other biological compounds. Other embodiments of the invention can have the drug or additive (such as a GRAS compound) be absorbed into the gastrointestinal tract and metabolized to release a volatile marker (such as a metabolite of the drug or additive) that is detectable in bodily fluids such as exhaled breath.

In certain embodiments, an additive is concurrently administered with a therapeutic drug, wherein the additive is the therapeutic drug marker. In one embodiment, medication comprising the therapeutic agent also includes the additive. For example, a pill can be manufactured that comprises the therapeutic drug and additive, or the additive can be provided in the pill coating or a solution of suspension of the therapeutic drug.

In other embodiments, the additive is taken separately in some form with the therapeutic drug to provide a convenient and non-invasive means for determining if the drug was taken under patient volitional action as prescribed (for example, by analyzing a sample of the patient's exhaled breath). For example, a patient may take two pills, wherein the first pill consists of the therapeutic drug and the second pill consists of the therapeutic drug marker.

When a therapeutic agent is taken by a patient and a corresponding therapeutic drug marker is subsequently presented in the patient, the preferred embodiment of the invention detects the presence of that therapeutic drug marker almost immediately in the exhaled breath of the patient (or possibly by requesting the patient to deliberately produce a burp) using an electronic nose of the invention.

In one embodiment, the therapeutic drug marker will coat the oral cavity or esophagus or stomach for a short while and be exhaled in the breath (or in a burp). For drugs administered in the form of pills, capsules, and fast-dissolving tablets, the markers can be applied as coatings or physically combined or added to therapeutic drug. Markers can also be included with therapeutic drugs that are administered in liquid form (i.e., syrups, via inhalers, or other dosing means). Certain therapeutic drug medications can have a coating to prevent the therapeutic drug marker from dissolving in the stomach and enable detection of the marker in exhaled breath.

In another embodiment, the therapeutic drug marker is an olfactory marker or a volatile organic marker such as a GRAS compound, that is concurrently administered with a therapeutic drug (e.g., the therapeutic marker is added to the coating of the pill or in a separate fast dissolving compartment in the pill or the solution if the medication is in liquid or suspension form) to provide a method for determining if the drug was taken by the patient as prescribed.

In another embodiment, a metabolite of a therapeutic drug (for example, where a metabolite of a therapeutic drug is released upon interaction with stomach acid or an enzyme) may be detected in exhaled breath to determine patient compliance in taking a medication as prescribed.

In yet another embodiment, an additive is concurrently administered with a therapeutic drug, where the additive is metabolized to provide a detectable therapeutic marker (such as metabolite of the additive) in exhaled breath for assessment of patient compliance. Any number of benign compounds could be used as olfactory markers (such as volatile markers).

As noted earlier, therapeutic drug markers are detected by their physical and/or chemical properties, which does not preclude using the therapeutic drug itself as its own marker. In accordance with the present invention, therapeutic drug markers that are useful as an indication of therapeutic drug concentration in blood include the following olfactory markers, without limitation: dimethyl sulfoxide (DMSO), acetaldehyde, acetophenone, trans-Anethole (1-methoxy-4-propenyl benzene) (anise), benzaldehyde (benzoic aldehyde), benzyl alcohol, benzyl cinnamate, cadinene, camphene, camphor, cinnamaldehyde (3-phenylpropenal), garlic, citronellal, cresol, cyclohexane, eucalyptol, and eugenol, eugenyl methyl ether; butyl isobutyrate (n-butyl 2, methyl propanoate) (pineapple); citral (2-trans-3,7-dimethyl-2,6-actadiene-1-al); menthol (1-methyl-4-isopropylcyclohexane-3-ol); and α-Pinene (2,6,6-trimethylbicyclo-(3,1,1)-2-heptene). These markers are preferred since they are used in the food industry as flavor ingredients and are permitted by the Food and Drug Administration. As indicated above, olfactory markers for use in the present invention can be selected from a vast number of available compounds (see Fenaroli's Handbook of Flavor Ingredients, 4^{rd} edition, CRC Press, 2001) and use of such other applicable markers is contemplated herein.

The markers of the invention also include additives that have been federally approved and categorized as GRAS (“generally recognized as safe”), which are available on a database maintained by the U.S. Food and Drug Administration Center for Food Safety and Applied Nutrition. It is known that certain GRAS molecules have the ability to be transmitted to patient via a mucus membrane (such as gastrointestinal mucosa). Such GRAS compounds may be metabolized by an enzyme system of interest and generate a product or products that can be detected in exhaled breath. The following Table 1 provides a list of GRAS compounds that may be used in accordance with the subject invention. Additional GRAS compounds that are readily detectable in exhaled breath for use in the present invention include, but are not limited to, sodium bisulfate, dioctyl sodium sulfosuccinate, polyglycerol polyricinoleic acid, calcium casein peptone-calcium phosphate, botanicals (i.e., chrysanthemum; licorice; jellywort, honeysuckle; lophatherum, mulberry leaf; frangipani; selfheal; sophora flower bud), ferrous bisglycinate chelate, seaweed-derived calcium, DHASCO (docosahexaenoic acid-rich single-cell oil) and ARASCO (arachidonic acid-rich single-cell oil), fructooligosaccharide, trehalose, gamma cyclodextrin, phytosterol esters, gum arabic, potassium bisulfate, stearyl alcohol, erythritol, D-tagatose, and mycoprotein.

TABLE 1
List of Potential PMEs based on GRAS

| DOCTYPE | DOCNUM | MAINTERM | CODE* | REGNUM |
|---|---|---|---|---|
| ASP | 311 | Dibenzyl Ether | 000103- | 172.515 |
| | | | 50-4 | |
| ASP | 328 | Difurfuryl Ether | 004437- | |
| | | | 22-3 | |
| ASP | 445 | Ethylene Glycol | 000111- | 178.1010 |
| | | Monobutyl Ether | 76-2 | 175.105 |
| | | | | 176.210 |
| | | | | 177.1650 |
| ASP | 557 | Furfuryl Methyl Ether | 013679- | |
| | | | 46-4 | |
| ASP | 775 | Isoeugenyl Benzyl | 000120- | 172.515 |
| | | Ether | 11-6 | |
| ASP | 776 | Isoeugenyl Ethyl | 007784- | 172.515 |
| | | Ether | 67-0 | |
| ASP | 778 | Isoeugenyl Methyl | 000093- | 172.515 |
| | | Ether | 16-3 | |
| ASP | 1023 | Methyl Phenethyl | 003558- | |
| | | Ether | 60-9 | |
| ASP | 1097 | Beta-Naphthyl | 002173- | |
| | | Isobutyl Ether | 57-1 | |
| ASP | 3017 | Vanillyl Butyl Ether | 082654- | |
| | | | 98-6 | |
| NIL | 2028 | Dimethylethanolamine | 000108- | 173.20 |
| | | | 01-0 | 175.105 |
| | | | | 175.300 |
| EAF | 3383 | Isopentylideneiso- | 035448- | |
| | | pentylamine | 31-8 | |

*CAS, RN, OR OTHER CODE

The definitions of the labels that are provided in Table 1 are as follows:

| Label | Definition | |
|---|---|---|
| DOCTYPE | An indicator of the status of the toxicology information available for | |
| | the substance in PAFA (administrative and chemical information is | |
| | available on all substances) | |
| | ASP | Fully up-to-date toxicology information has been sought |
| | EAF | There is reported use of the substance, but it has not yet |
| | | been assigned for toxicology literature research |
| | NEW | There is reported use of the substance, and an initial |
| | | toxicology literature search is in progress |
| | NIL | Although listed as added to food, there is no current |
| | | reported use of the substance and, therefore, although |
| | | toxicology information may be available in PAFA, it is not |
| | | being updated |
| | NUL | There is no reported use of the substance and there is no |
| | | toxicology information available in PAFA |
| | BAN | The substance was formerly approved as a food additive but |
| | | is now banned; there may be some toxicology data available |

| | |
|---|---|
| DOCNUM | PAFA database number of the Food Additive Safety Profile volume |
| | containing the printed source information concerning the substance |
| MAINTERM | The name of the substance as recognized by CFSAN |
| CAS, RN, | Chemical Abstract Service (CAS) Registry Number for the substance |
| OR OTHER | or a numerical code assigned by CFSAN to those substances that do |
| CODE | not have a CAS Registry Number (888nnnnnn or 977nnnnn-series) |
| REGNUM | Regulation numbers in Title 21 of the U.S. Code of Federal |
| | Regulations where the chemical appears |

Certain markers of the invention are used for indicating specific drugs or for a class of drugs. For example, with a patient taking an antibiotic, an antihypertensive agent, and an anti-reflux drug, one marker is used for antibiotics as a class; alternatively, one marker is used to represent subclasses of antibiotics, such as erythromycins. Another marker could be used for antihypertensives as a class, or for specific subclasses of antihypertensives, such as calcium channel blockers. The same would be true for the anti-reflux drug. Furthermore, combinations of marker substances could be used allowing a rather small number of markers to specifically identify a large number of medications.

In one method of operation, an electronic nose is used to identify a baseline marker spectrum for the patient prior to administration of the medication. This will prove beneficial for the detection of more than one drug if the patient is required to ingest more than one drug at a time. Further, the baseline spectrum will enable identification of interference markers derived from different foods and odors in the stomach, mouth, esophagus and lungs.

In a preferred embodiment, the markers of the invention are either metabolites of the therapeutic drug or metabolites of an additive that is administered concurrently with the therapeutic drug. Preferably, the metabolites are detectable in exhaled breath using sensors as described herein to provide notice of patient compliance in taking the therapeutic drug.

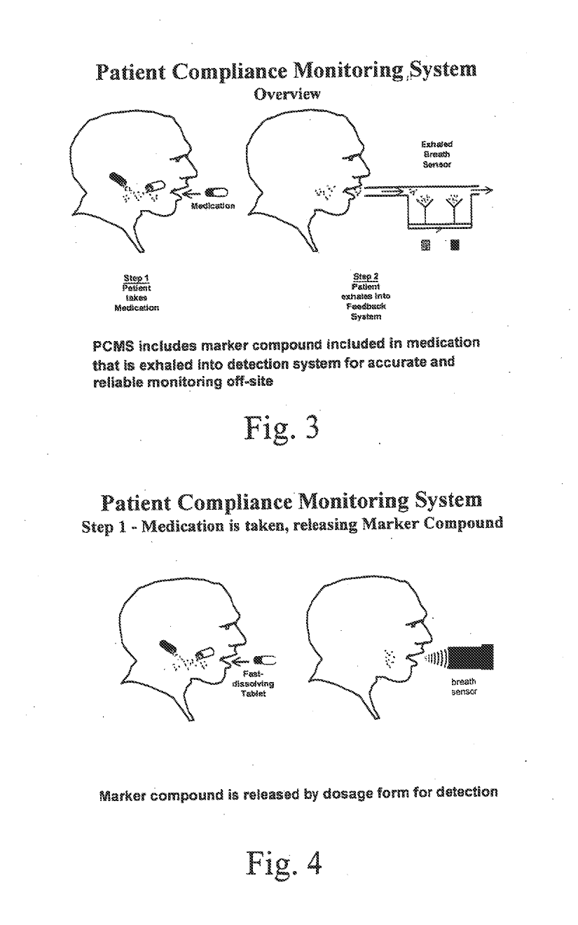

When the drugs or drugs including markers/additives are taken (FIG. 3), the drugs are dissolved in the mouth (or digested in the stomach, or absorbed into the gastro-intestinal tract and metabolized, or transmitted to the lungs, etc.). The sensor of the invention such as an electronic nose can then detect therapeutic drug marker(s) associated with the drug(s) when the patient exhales (FIGS. 3-5) to confirm that the medication was taken by patient volitional action, even on a dose by dose basis. The electronic nose can record and/or transmit the data sensed from the patient's breath for monitoring purposes.

For example, in accordance with the subject invention, an electronic nose is used to analyze the patient's breath for detectable metabolites of a therapeutic agent to determine patient volitional action in taking a therapeutic agent as prescribed. Alternatively, an electronic nose is used to analyze the patient's breath for detectable metabolites of an additive that was administered concurrently with a therapeutic drug. Detection of the additive metabolite(s) would confirm patient volitional action in taking the therapeutic drug.

In one embodiment, a therapeutic drug marker is concurrently administered with a therapeutic drug (i.e., marker is provided in a pharmaceutically acceptable carrier—marker in medication coating composed of rapidly dissolving glucose and/or sucrose). In a related embodiment, the therapeutic drug is provided in a form for oral ingestion (such as in the form of a pill), whose coating includes at least one marker in air-flocculated sugar crystals. This would stimulate salivation and serve to spread the marker around the oral cavity, enhancing the lifetime in the cavity. Since the throat and esophagus could also be coated with the marker as the medication is ingested, detection of the marker is further enhanced.

While the primary goal of the invention is to improve and document medication compliance in motivated, responsible (albeit occasionally forgetful) individuals, there is a small minority of patients who intentionally do not take their medications, or whose failure to take their medication can result in a public health crisis (i.e. the spread of drug resistant tuberculosis). As a further guarantee that these individuals do not use deceptive practices to “fool” the sensors (i.e. dissolving the tablet or capsules in a small amount of water to release the marker), a pressure sensor can be incorporated into the detector to document that the patient is actually exhaling through the device. A flow restrictor can be incorporated which increases the resistance to exhalation. By the simple addition of a pressure transducer to the system, a pressure change from baseline can be measured during exhalation. Additionally, a number of detectors are available (i.e. end-tidal carbon dioxide monitors) that can be added to the device for use in environments where deception may be likely (i.e. institutions and prisons) and the consequences severe.

Additional embodiments are also envisioned herein. Pulmonary delivery of medications is well known, especially for conditions such as asthma and chronic obstructive pulmonary disease. In these instances, medication (i.e. corticosteroids, bronchodilators, anticholenergics, etc.) is often nebulized or aerosolized and inhaled through the mouth directly into the lungs. This allows delivery directly to the affected organ (the lungs) and reduces side effects common with enteral (oral) delivery. Metered dose inhalers (MDIs) or nebulizers are commonly used to deliver medication by this route. Recently dry powder inhalers have become increasingly popular, as they do not require the use of propellants such as CFCs. Propellants have been implicated in worsening asthma attacks, as well as depleting the ozone layer. Dry power inhalers are also being used for drugs that were previously given only by other routes, such as insulin, peptides, and hormones.

Therapeutic drug markers, in particular olfactory markers or additives whose metabolites are detectable in exhaled breath, can be added to these delivery systems as well. Since the devices are designed to deliver medication by the pulmonary route, the sensor array can be incorporated into the device and the patient need only exhale back through the device for documentation to occur.

Lastly, devices are available to deliver medication by the intranasal route. This route is often used for patients with viral infections or allergic rhinitis, but is being increasing used to deliver peptides and hormones as well. Again, it would be simple to incorporate a sensor array into these devices, or the patient can exhale through the nose for detection by a marker sensing system.

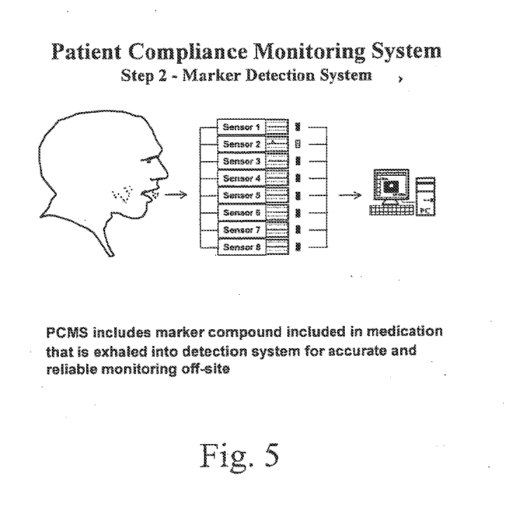

The electronic nose and/or computer communicating therewith (FIG. 5) can also notify the medical staff and/or the patient to any irregularities in dosing, dangerous drug interactions, and the like. This system will enable determination as to whether a patient has taken the prescribed drug at the appropriate time and at the prescribed dosage. The device could also alert the patient that it is time to take their medications.

### Remote Communication System

A further embodiment of the invention includes a communications device in the home (or other remote location) that will be interfaced to the sensor. The home communications device will be able to transmit immediately or at prescribed intervals directly or over a standard telephone line (or other communication transmittal means such as satellite transmission, via the internet, etc.) the data collected by the data monitor/analyzer device. The communication of the data will allow the user (i.e., physician) to be able to remotely verify if the appropriate dosage of a therapeutic drug is being administered to the patient. The data transmitted from the home can also be downloaded to a computer where the drug blood levels are stored in a database, and any deviations outside of pharmacological efficacy would be automatically flagged (i.e., alarm) so that a user (i.e., patient, physician, nurse) could appropriately adjust the drug dosage per suggestions provided by a computer processing unit connected to the sensor or per dosage suggestions provided by health care personnel (i.e., physician).

### Therapeutic Drugs

Therapeutic drugs that can be monitored in accordance with the subject invention to assess patient compliance with a therapeutic drug regimen include, but are not limited to, the following: α-Hydroxy-Alprazolam; Acecamide (NAPA); Acetaminophen (Tylenol); Acetylmorphine; Acetylsalicylic Acid (as Salicylates); α-hydroxy-alprazolam; Alprazolam (Xanax); Amantadine (Symmetrel); Ambien (Zolpidem); Amikacin (Amikin); Amiodarone (Cordarone); Amitriptyline (Elavil) & Nortriptyline; Amobarbital (Amytal); Anafranil (Clomipramine) & Desmethylclomipramine; Ativan (Lorazepam); Aventyl (Nortriptyline); Benadryl (Dephenhydramine); Benziodiazepines; Benzoylecgonine; Benztropine (Cogentin); Bupivacaine (Marcaine); Bupropion (Wellbutrin) and Hydroxybupropion; Butabarbital (Butisol); Butalbital (Fiorinal) Carbamazepine (Tegretol); Cardizem (Diltiazem); Carisoprodol (Soma) & Meprobamate; and Celexa (Citalopram & Desmethylcitalopram).

Additional therapeutic drugs that can be monitored in accordance with the subject invention include Celontin (Methsuximide) (as desmethylmethsuximide); Centrax (Prazepam) (as Desmethyldiazepam); Chloramphenicol (Chloromycetin); Chlordiazepoxide; Chlorpromazine (Thorazine); Chlorpropamide (Diabinese); Clonazepam (Klonopin); Clorazepate (Tranxene); Clozapine; Cocaethylene; Codeine; Cogentin (Benztropine); Compazine (Prochlorperazine); Cordarone (Amiodarone); Coumadin (Warfarin); Cyclobenzaprine (Flexeril); Cyclosporine (Sandimmune); Cylert (Pemoline); Dalmane (Flurazepam) & Desalkylflurazepam; Darvocet; Darvon (Propoxyphene) & Norpropoxyphene; Demerol (Meperidine) & Normeperidine; Depakene (Valproic Acid); Depakote (Divalproex) (Measured as Valproic Acid); Desipramine (Norpramin); Desmethyldiazepam; Desyrel (Trazodone); Diazepam & Desmethyldiazepam; Diazepam (Valium) Desmethyldiazepam; Dieldrin; Digoxin (Lanoxin); Dilantin (Phenyloin); Disopyramide (Norpace); Dolophine (Methadone); Doriden (Glutethimide); Doxepin (Sinequan) and Desmethyldoxepin; Effexor (Venlafaxine); Ephedrine; Equanil (Meprobamate) Ethanol; Ethosuximide (Zarontin); Ethotoin (Peganone); Felbamate (Felbatol); Fentanyl (Innovar); Fioricet; Fipronil; Flunitrazepam (Rohypnol); Fluoxetine (Prozac) & Norfluoxetine; Fluphenazine (Prolixin); Fluvoxamine (Luvox); Gabapentin (Neurontin); Gamma-Hydroxybutyric Acid (GHB); Garamycin (Gentamicin); Gentamicin (Garamycin); Halazepam (Paxipam); Halcion (Triazolam); Haldol (Haloperidol); Hydrocodone (Hycodan); Hydroxyzine (Vistaril); Ibuprofen (Advil, Motrin, Nuprin, Rufen); Imipramine (Tofranil) and Desipramine; Inderal (Propranolol); Keppra (Levetiracetam); Ketamine; Lamotrigine (Lamictal); Lanoxin (Digoxin); Lidocaine (Xylocalne); Lindane (Gamma-BHC); Lithium; Lopressor (Metoprolol); Lorazepam (Ativan); and Ludiomil.

The presence and/or concentration of the following therapeutic drugs that can be monitored in accordance with the subject invention include, but are not limited to, (Maprotiline); Mebaral (Mephobarbital) & Phenobarbital; Mellaril (Thioridazine) & Mesoridazine; Mephenyloin (Mesantoin); Meprobarnate (Miltown, Equanil); Mesantoin (Mephenyloin); Mesoridazine (Serentil); Methadone; Methotrexate (Mexate); Methsuximide (Celontin) (as desmethsuximide); Mexiletine (Mexitil); Midazolam (Versed); Mirtazapine (Remeron); Mogadone (Nitrazepam); Molindone (Moban); Morphine; Mysoline (Primidone) & Phenobarbital; NAPA & Procainamide (Pronestyl); NAPA (N-Acetyl-Procainamide); Navane (Thiothixene); Nebcin (Tobramycin); Nefazodone (Serzone); Nembutal (Pentobarbital); Nordiazepam; Olanzapine (Zyprexa); Opiates; Orinase (Tolbutamide); Oxazepam (Serax); Oxcarbazepine (Trileptal) as 10-Hydroxyoxcarbazepine; Oxycodone (Percodan); Oxymorphone (Numorphan); Pamelor (Nortriptyline); Paroxetine (Paxil); Paxil (Paroxetine); Paxipam (Halazepam); Peganone (Ethotoin); PEMA (Phenylethylmalonamide); Pentothal (Thiopental); Perphenazine (Trilafon); Phenergan (Promethazine); Phenothiazine; Phentermine; Phenylglyoxylic Acid; Procainamide (Pronestyl) & NAPA; Promazine (Sparine); Propafenone (Rythmol); Protriptyline (Vivactyl); Pseudoephedrine; Quetiapine (Seroquel); Restoril (Temazepam); Risperdal (Risperidone) and Hydroxyrisperidone; Secobarbital (Seconal); Sertraline (Zoloft) & Desmethylsertraline; Stelazine (Trifluoperazine); Surmontil (Trimipramine); Tocainide (Tonocard); and Topamax (Topiramate).

The therapeutic drugs of the invention (as well as therapeutic drug markers) can be used in a variety of routes of administration, including, for example, orally-administrable forms such as tablets or liquid medications, capsules or the like, or via parenteral, intravenous, intramuscular, transdermal, buccal, subcutaneous, suppository, or other route. Such compositions are referred to herein generically as “pharmaceutical compositions.” Typically, they can be in unit dosage form, namely, in physically discrete units suitable as unitary dosages for human consumption, each unit containing a predetermined quantity of active ingredient calculated to produce the desired therapeutic effect in association with one or more pharmaceutically acceptable other ingredients, i.e., diluent or carrier.

### Example 1

### Metabolite of Verapamil as a Therapeutic Drug Marker

Verapamil is a widely prescribed calcium channel antagonist used mainly to treat essential hypertension or cardiac arrhythmias. The metabolism of verapamil is via the cytochrome P450 3A4 system that metabolizes many drugs by oxidative N-dealkylation. It is commonly observed that the alkyl group lost from an amine during N-dealkylation (and from an ether during O-dealkylation) appears as an aldehyde or ketone arising from the dissociation of a carbinolamine intermediate (Brodie et al., “Enzymatic metabolism of drugs and other foreign compounds,” Annu Rev. Biochem, 27:427-454 (1958); Rose and Castagnoli, “The metabolism of tertiary-amines,” Med Res Rev., 3(1):73-88 (1983)).

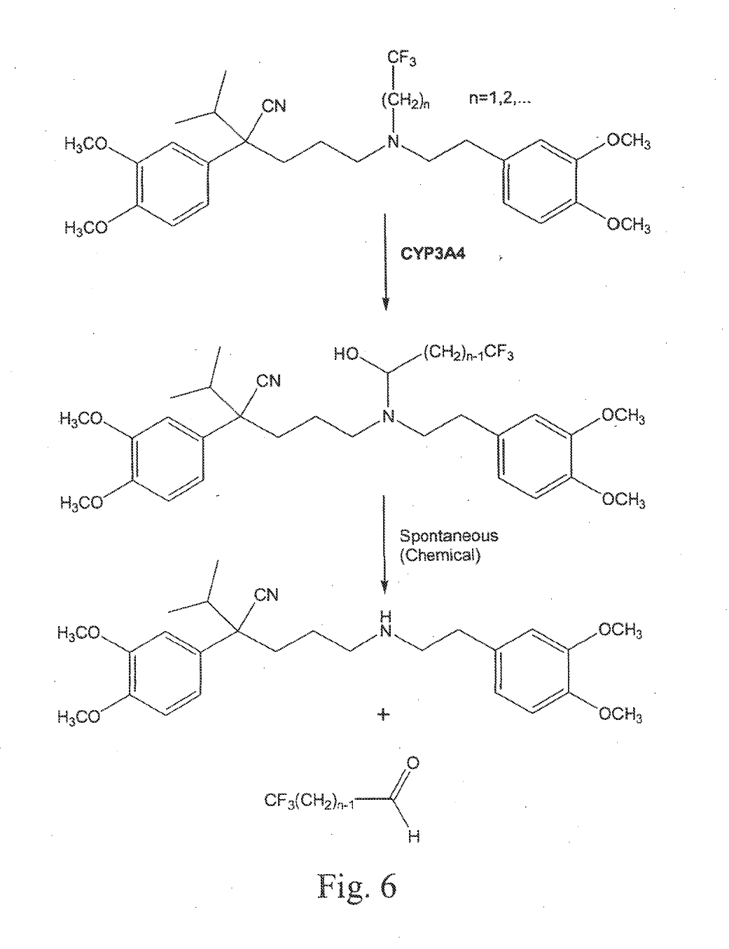

In this particular example of the present technology, verapamil is modified so that instead of the native formaldehyde being liberated due to metabolism, a non-endogenous volatile molecule is produced in a 1:1 molar ratio to the parent substrate (see FIG. 6). This volatile product is transported to the lungs and excreted into the alveolar space. During exhalation, nearly all pulmonary gas is expired (except residual volume) so that a significant mass of the volatile marker can be detected in exhaled breath using various measurement methodologies (for example, SAW, infrared, fuel cell, etc.).

Verapamil undergoes an extensive hepatic metabolism. Due to a large hepatic first-pass effect, bioavailability does not exceed 20-35% in normal subjects. Twelve metabolites have been described. The main metabolite is norverapamil and the others are various N- and O-dealkylated metabolites (Knoll Pharmaceuticals, Product Information: Isoptin SR (1984); Shomerus et al., “Physiological disposition of verapamil in man,” Cardiovasc Res., 10:605-612 (1976)). In accordance with the subject invention, the metabolites of verapamil, such as norverapamil and others, that are produced after administration of verapamil to a patient represent therapeutic drug markers can be detected using sensors of the invention, which indicate patient compliance in taking verapamil as prescribed.

### Example 1A

### Synthesis of a Verapamil Analogue that Produces a Detectable Metabolite after Administration to a Patient

N-Nor-(+)-verapamil hydrochloride (477 mg, 1 mmol) is suspended in 10 mL methanol, and sodium hydroxide (40 mg, 1 mmol) is added. The precipitate is filtered off; then, the solvent is evaporated in vacuo. The residue is dissolved in acetonitrile (10 mL), polystyrene-bound 1,5,7-triazabicyclo[4,4,0]dec-5-ene (2 g) and 3-bromo-1,1,1-trifluoropropane (195 mg, 1.1 mmol) are added to the solution. The mixture is stirred at room temperature for 16 h. Scavenger resin (methylisocyanate bound to macroporous polystyrene resin, 2 g) is then added and the reaction mixture is agitated for a further 16 h. The solid is filtered off, washed with acetonitrile (2×5 mL), the filtrate is evaporated to dryness in vacuo, and the residue is purified by silica gel column chromatography. The purified product is then treated with diethyl ether containing 2M hydrochloric acid to obtain it in a salt form.

### Example 2

### Metabolite of Dextromethorphan as a Therapeutic Marker

Dextromethorphan (3 Methoxy-17-methylmorphinan hydrobromide monohydrate; MW 370.3) is the d isomer of levophenol, a codeine analogue and opioid analgesic. The main clinical use of this agent is as an antitussive.

There is a clear first pass metabolism of dextromethorphan. It is generally assumed that the therapeutic activity of dextromethorphan is primarily due to its active metabolite, dextrophan (Silvasti et al., “Pharmacokinetics of dextromethorphan and dextrorphan: a single dose comparison of three preparations in human volunteers, Int J Clin Pharmacol Ther Toxicol, 9:493-497 (1987); Baselt & Cravey, Disposition of Toxic Drugs and Chemicals in Man, 3^{rd} ed. Yearbook Medical Publishers, Inc., Chicago (1982)). It is metabolized in the liver by extensive metabolizers to dextrorphan. Dextrorphan is itself an active antitussive compound (Baselt & Cravey, 1982). Only small amounts are formed in poor metabolizers (Kupfer et al., “Pharmacogenetics of dextromethorphan O-demethylation in man,” Xenobiotica, 16:421-433 (1986)). Less than 15% of the dose form minor metabolites including D-methoxymorphinane and D-hydroxmorphinane (Kupfer et al., 1986).

### Example 2a

### Synthesis of Dextromethorphan Analogue that Produces a Detectable Metabolite after Administration to a Patient

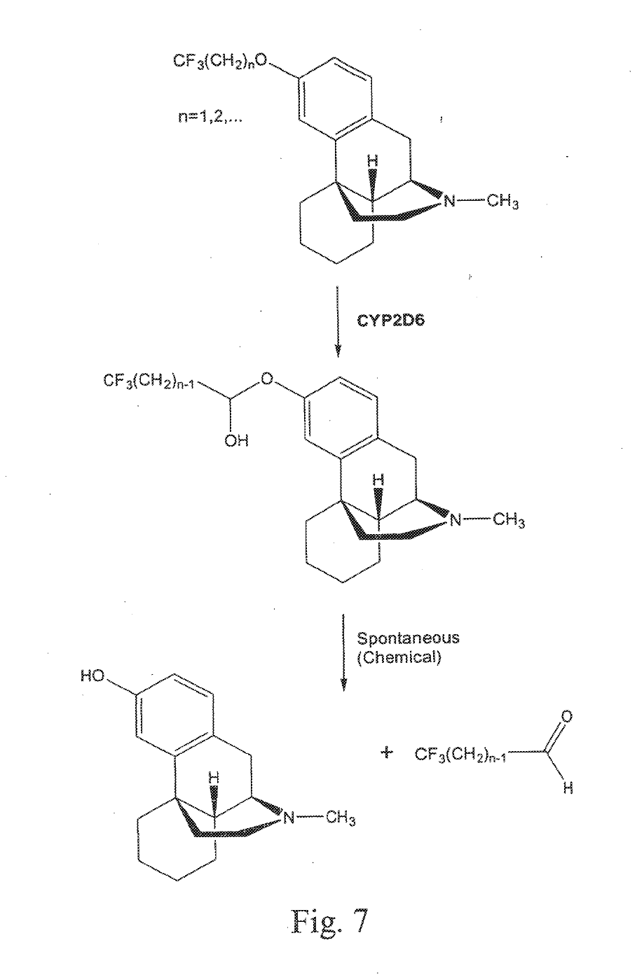

Using a similar method to Example 1A above (verapamil modification), dextromethorphan is modified to form a product that when metabolized produces a therapeutic marker that is detectable in exhaled breath of humans (see FIG. 7). 3-Hydroxy-N-methylmorphinan tartrate salt (407 mg, 1 mmol) is dissolved in water (2 mL) and treated with saturated potassius carbonate solution until pH 10 is reached. The aqueous solution is extracted with chloroform (3×2 mL), the combined organic extract is dried over anhydrous sodium carbonate, and the solvent is evaporated at room temperature under nitrogen stream. Under dry nitrogen atmosphere, the residue is dissolved in 5 ml of dry 1,2-dichloroethane at room temperature. 1,8-Bis(dimethylamino)naphthalene (Proton Sponge, 215 mg, 1.2 mmol) and vinyl chloroformate (245 mg, 2.2 mmol) are added, and the solution is heated overnight at 60° C. The solvent is evaporated in vacuo, and the residue is purified by silica gel column chromatography (dichloromethane as an eluent). After evaporation of the solvent in vacuo, the vinyloxycarbonyl-protected 3-hydroxymorphinan is treated with dioxane (6 mL) and water (2 mL) containing 40 mg (1 mmol) sodium hydroxide, and the solution is heated at 50° C. for 4 h. The mixture is cooled to room temperature, poured into brine and, then, extracted with 3×10 mL diethyl ether. The combined extract is dried over sodium sulfate, and the solvent is evaporated in vacuo. The residue is dissolved in tetrahydrofuran (5 mL), polystyrene-bound 1,5,7-triazabicyclo[4,4,0]dec-5-ene (2 g) and 3-bromo-1,1,1-trifluoropropane (195 mg, 1.1 mmol) are added to the solution. After stiffing at room temperature for 2 h, the solid is filtered off, washed with tetrahydrofuran (2×2 mL), and the combined filtrate is evaporated to dryness in vacuo. The residue is dissolved in diethyl ether (1 mL) and treated with aqueous 47% hydrogen bromide (0.1 mL). The resulting solid is filtered off and recrystallized from diethyl ether.

Other therapeutic drug systems amenable to this type of competency examination include, but are not limited to, alcohol dehydrogenase, alkaline phosphatase, sulfatase, cholinesterase, glucose-6-phosphate dehydrogenase, prostate specific antigen and others.

It should be understood that the examples and embodiments described herein are for illustrative purposes only and that various modifications or changes in light thereof will be suggested to persons skilled in the art and are to be included within the spirit and purview of this application and the scope of the appended claims. Specifically, the marker detection method of the present invention is intended to cover detection not only through the exhalation by a patient with a device utilizing electronic nose technology, but also other suitable technologies, such as gas chromatography, transcutaneous/transdermal detection, semiconductive gas sensors, mass spectrometers, IR or UV or visible or fluorescence spectrophotometers.

All patents, patent applications, provisional applications, and publications referred to or cited herein, or from which a claim for benefit of priority has been made, are incorporated by reference in their entirety to the extent they are not inconsistent with the explicit teachings of this specification

We claim:

1. A device or system for non-invasively determining patient compliance in taking medication, said medication comprising at least one therapeutic drug and an additive selected such that the additive or a metabolite of the additive or both are known to be detectable in exhaled breath as a therapeutic drug marker by a sensor selected for that purpose when said medication is taken by said patient, said system comprising:
(a) a sensor for analyzing a sample of the patient's breath to measure the therapeutic drug marker in the patient's breath, wherein the therapeutic drug marker is detectable in the patient's breath after taking the medication combined with additive;
(b) an indicator of patient compliance or non-compliance in taking the medication coupled with said sensor such that when said marker is detected in the patient's breath, the indicator provides an indication of compliance and if said marker is not detected, said indicator provides an indication of non-compliance; and
(c) a flow sensor to detect starting and completion of exhalation.

2. The device or system according to claim 1 wherein said sensor comprises a gas chromatography sensor.

3. The device or system according to claim 1 wherein said sensor is in operative communication with a CPU as a data processing/control unit for either or both:
(i) automatically detecting the signal from said flow sensor to control sampling of exhaled breath;
(ii) providing to the user/patient the appropriate dosage of the therapeutic drug to be delivered or information on whether the patient has taken the prescribed medication at the correct time and dosage from analysis of trends in therapeutic drug blood concentration based on known pharmacokinetic and pharmacodynamic properties of the selected therapeutic drug marker's appearance in the exhaled breath.

4. The device or system according to claim 1 further comprising any one, a combination or all of the following components:
a. a selector for sampling of end-tidal exhaled breath;
b. said flow sensor configured to detect starting and completion of exhalation;
c. data processing/control unit for automatically detecting the signal from a flow sensor to control sampling of exhaled breath;
d. a CPU which provides appropriate dosage of therapeutic drug to be delivered based on analysis of trends in said marker detected in exhaled breath;
e. a gas chromatographic column for separating components of exhaled breath;
f. a container adapted for capture of exhaled breath for analysis by said sensor;
g. an exhaled breath sample dehumidifier prior of said sensor to said exhaled breath sample.

5. The device or system according to claim 1 comprising a sensor configured to detect a GRAS therapeutic drug marker.

6. The device or system according to claim 5 wherein said GRAS marker is selected from the group consisting of sodium bisulfate; dioctyl sodium sulfosuccinate; polyglycerol polyricinoleic acid; calcium casein peptone-calcium phosphate; chrysanthemum; licorice; jellywort, honeysuckle; lophatherum, mulberry leaf; frangipani; selfheal; sophora flower bud; ferrous bisglycinate chelate; seaweed-derived calcium; docosahexaenoic acid-rich single-cell oil; arachidonic acid-rich single-cell oil; fructooligosaccharide; trehalose; gamma cyclodextrin; phytosterol esters; gum arabic; potassium bisulfate; stearyl alcohol; erythritol; D-tagatose; mycoprotein; dimethyl sulfoxide (DMSO), acetaldehyde, acetophenone, trans-Anethole (1-methoxy-4-propenyl benzene) (anise), benzaldehyde (benzoic aldehyde), benzyl alcohol, benzyl cinnamate, cadinene, camphene, camphor, cinnamaldehyde (3-phenylpropenal), garlic, citronellal, cresol, cyclohexane, eucalyptol, and eugenol, eugenyl methyl ether; butyl isobutyrate (n-butyl 2, methyl propanoate) (pineapple); citral (2-trans-3,7-dimethyl-2,6-actadiene-1-al); menthol (1-methyl-4-isopropylcyclohexane-3-ol); and α-Pinene (2,6,6-trimethylbicyclo-(3,1,1)-2-heptene).

7. The device or system according to claim 1 comprising a sensor selected from the group consisting of metal-insulator-metal ensemble (MIME) sensors; cross-reactive optical microsensor arrays; fluorescent polymer films; surface enhanced raman spectroscopy (SERS); diode lasers; selected ion flow tubes; metal oxide sensors (MOS); bulk acoustic wave (BAW) sensors; colorimetric tubes; infrared spectroscopy; gas chromatography; semiconductive gas sensor technology; mass spectrometers; fluorescent spectrophotometers; conductive polymer gas sensor technology; aptamer sensor technology; amplifying fluorescent polymer (AFP) sensor technology; photo-ionization detectors, and ion mobility spectrometry technology.

8. The device or system according to claim 1 comprising a sensor which produces a unique electronic fingerprint characterizing the marker such that the presence, concentration and fluctuations in the concentration of the marker is determined.

9. The device or system according to claim 1 adapted to record data resulting from analysis of the patient's breath.

10. The device or system according to claim 9 adapted to transmit data resulting from the analysis of the patient's breath.

11. The device or system according to claim 1 adapted to analyze the patient's breath by comparing the marker sensed in the patient's breath with a predetermined signature profile of a specific marker.

12. The device or system according to claim 1 wherein said predetermined signature profile of a specific marker is associated with a class of drugs.

13. The device or system according to claim 1 adapted to measure a baseline marker spectrum for the patient prior to the patient's taking of the medication.

14. The device or system according to claim 1 adapted to detect said marker in a specific phase of the exhaled breath.

15. The device or system according to claim 14 wherein said specific phase is the end tidal breath phase or the end tidal pCO2 phase of the exhaled breath.

16. The device or system according to claim 1 wherein said flow sensor detects starting and/or completion of exhalation.

17. The device or system according to claim 3 wherein said CPU detects a signal from said flow sensor to control sampling of exhaled breath.

18. The device or system according to claim 1 wherein said additive, a metabolite of said additive, or both is detected as the therapeutic drug marker.

19. The device or system according to claim 1 further comprising a pressure sensor which measures a pressure level at the step of obtaining a sample of the patient's breath, in which the pressure level is checked against a threshold in order to determine that the patient has expelled breath.

20. The device or system according to claim 1 said sensor providing a detection signal which varies in relation to the concentration of the therapeutic drug marker in the patient's breath.

21. The system according to claim 1 comprising a device according to claim 1 used in combination with a medication comprising an additive which produces an exhaled drug marker after being taken or administered by a patient.

22. The system according to claim 21 comprising providing the therapeutic drug in the form of: a liquid medication for oral administration; a pulmonary delivered medication; an intranasal delivered medication; an intravenously delivered medication

23. The system according to claim 21 wherein said additive is provided with a therapeutic agent as a pill coating, as a solution in suspension with said therapeutic drug, or as a separate fast dissolving compartment in a pill.

24. The system according to claim 21 wherein said medication is dissolved in the mouth, digested in the stomach, or absorbed into the gastrointestinal tract and metabolized, or is transmitted to the lungs.

25. The system according to claim 21 wherein said therapeutic drug marker is provided with said therapeutic drug in a pharmaceutically acceptable carrier coating composed of rapidly dissolving glucose and/or sucrose.

26. The system according to claim 21 wherein said therapeutic drug is provided in a form for oral ingestion the coating of which includes at least one marker in air-flocculated sugar crystals.

27. The system according to claim 26 wherein said form stimulates salivation and serves to spread the marker around the oral cavity, enhancing the lifetime in the cavity and coating the throat and esophagus.
